(19) United States
(12) Patent Application Publication
(10) Pub. No.: US 2003/0120327 A1
(43) Pub. Date: Jun. 26, 2003

(54) Medical lead adaptor assembly with retainer

(76) Inventors: Mark Tobritzhofer, Fridley, MN (US); Dwight Lauinger, Hutchinson, MN (US); Al Knudson, Zimmerman, MN (US); Greg Larson, Mahtomedi, MN (US); Eric Huff, St. Paul, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/033,816

(22) Filed: Dec. 20, 2001

(51) Int. Cl. A61N 1/04

(52) U.S. Cl. 607/116; 607/37

(57) ABSTRACT

A medical lead adaptor assembly for making an electrical connection between a cardiac lead and an external medical device, with the cardiac lead including a lead connector pin for electrically coupling the cardiac lead to the lead adaptor assembly. The lead adaptor assembly includes a connector end assembly that electrically couples the lead adaptor assembly to the external device, a housing portion that has a lead receptacle portion including a lead receptacle that receives the lead connector pin of the cardiac lead to electrically couple the cardiac lead to the lead adaptor assembly, and a retaining flange that extends from the housing portion and has a retaining slot that receives and retains the cardiac lead to substantially prevent corruption of the electrical coupling of the cardiac lead to the lead adaptor assembly.

### FIELD OF THE INVENTION

The present invention generally relates to a medical lead adaptor assembly, and in particular, the present invention relates to a medical lead adaptor assembly for making a temporary connection between a medical lead of an implantable medical device and an external medical device.

### BACKGROUND OF THE INVENTION

The earliest instances of relatively prolonged cardiac stimulation, namely cardiac pacing, of a patient's heart was effected through implanted cardiac leads attached to the heart muscle at distal electrode ends and extended through an incision in the patient's skin. Initially, cardiac pacing was employed during postoperative recovery from cardiac surgery, and the attachment to the heart was made to the epicardium during the surgical procedure. To effect unipolar pacing of the heart, a single such implantable pacing lead was employed in conjunction with a subcutaneously implanted or skin surface attached return electrode coupled to an external lead conductor. To effect bipolar pacing of the heart, two such implantable pacing leads were implanted with the electrode ends implanted a distance apart. Initially, the attachment mechanism typically required a second surgical procedure to remove the distal electrode(s) and the pacing lead(s).

The attachment of the proximal ends of the lead conductors to the temporary cardiac pacemaker connector elements was initially effected by simply stripping insulation from the proximal conductor ends, inserting the bare conductor ends around or through transverse openings in threaded posts, and tightening down thumb nuts. Later, finished connector pins were formed at the proximal connector ends of the lead bodies that could be inserted into end openings of the thumb nuts and connector posts.

Implantable pacing leads evolved into permanent, unipolar and bipolar, endocardial and epicardial, pacing leads for chronic implantation in a patient and with proximal electrical connector assemblies connected with connector elements of a totally implanted, cardiac pacemaker pulse generator. To withstand stress, implantable pacing lead conductors were formed of coiled wire and inserted within an insulative lead body lumen, thereby providing a coiled wire lumen that was sized to receive a stiffening stylet wire to assist transvenous implantation of endocardial pacing leads. The proximal end of the coiled wire conductor was attached to a tubular connector pin at the terminus of the lead connector end shaped to be received in the connector assembly of the implantable pacemaker pulse generator. In the case of endocardial permanent pacing leads, the connector pin was formed with a lumen therein aligned with the coiled wire lumen so that the stiffening stylet wire could be inserted down the length of the lead body, used during the transvenous introduction, and withdrawn after placement of the distal electrode was achieved. Many of these features are employed in current permanent pacing leads.

More recently, bipolar and multi-polar permanently implantable pacing leads and leads for use in pacing and cardioversion/defibrillation (collectively referred to as permanent implantable cardiac leads) have been developed using coaxially arranged, coiled wire conductors and/or parallel-wound, multi-filar coiled wire conductors. In the case of endocardial cardiac leads, the stylet wire lumen is employed to receive the stiffening stylet wire for implantation as described above. The proximal connector end assemblies are formed with at least two spaced apart lead connector elements arranged in-line from a proximal lead connector pin to at least one more distally located ring-shaped element or lead connector ring. Typical bipolar in-line lead connector assemblies for multi-filar, coiled wire conductors are shown, for example, in commonly assigned U.S. Pat. Nos. 4,944,088 and 4,951,687 and 5,007,435, respectively, incorporated herein by reference.

Different manufacturers have produced implantable cardiac leads with lead connector end assemblies that match the connector block terminals of implantable medical devices of the same manufacturer. In recent years, one dimensional pacemaker connector standard has been made implemented, namely the low profile connector “IS-1” standard (ISO 5841-3:1992(E)) for bipolar in-line and unipolar lead connector end assemblies. Other permanent, bipolar, in-line, cardiac lead connector end assemblies conform dimensionally with the MEDTRONIC 3.2 mm low profile connector standard. Certain permanent unipolar cardiac lead connector end assemblies conform dimensionally with the MEDTRONIC 5 mm connector standard.

Unipolar and bipolar, temporary endocardial pacing leads and temporary epicardial heart wires were also developed for implantation of the distal electrode(s) thereof in contact with the endocardium or sutured through the epicardium of the hearts of hospitalized patients. The lead body size of these temporary pacing leads and heart wires has typically been smaller than that of permanent cardiac leads because of the absence of an internal wire coil lumen for receiving a stiffening stylet wire. Still, in the case of bipolar temporary pacing leads and heart wires, either a lead connector pin and ring set having comparable separations apart to those of permanent cardiac leads or bifurcated lead connector assemblies are employed providing a pair of lead connector pins. Exemplary temporary bipolar pacing leads include the MEDTRONIC® TEMPTRON temporary pacing leads having a uniform diameter, in-line, connector pin and ring assembly. Exemplary heart wires include the MEDTRONIC® Model Nos. 6491, 6492, 6494 and 6500 unipolar heart wires and the Model 6495 bipolar heart wire as described in commonly assigned U.S. Pat. No. 4,341,226, incorporated herein by reference.

During a hospitalization, a heart wire or temporary pacing lead of these types may be implanted to allow monitoring and demand pacing of the heart as the patient recovers from cardiac surgery or another condition. In addition, it may be necessary at times to connect the proximal connector end of a permanent cardiac lead already implanted in a patient to a temporary external pacemaker. The proximal connector end assemblies in each case are attached, for example, to external medical device connector elements of MEDTRONIC® Model Nos. 5348 or 5388 external single chamber or dual chamber cardiac pacemakers. The external medical device connector elements of such external cardiac pacemakers can constitute either spaced RCA type female sockets or a shrouded connector housing that are not compatible with cardiac lead connector end assemblies.

Therefore, when a bipolar heart wire or permanent or temporary pacing lead is to be connected, it is necessary to use a further “patient cable” adaptor to complete the connection. The MEDTRONIC® Model 5433A/V or the Model 5832/S reusable safety cables are employed to make the connection between the temporary pacemaker and the proximal connector ends of the heart wire or temporary pacing lead. Alternatively, the MEDTRONIC® Model 5487/S or the Model 5833/S disposable cable is employed to make the connection between the temporary pacemaker and the proximal connector ends of a temporary pacing lead.

A similar situation arises during a surgical implantation of a pacemaker or pacemaker-cardioverter-defibrillator including a permanent cardiac lead or lead system or the replacement connection of a implantable pulse generator of one of these types with a pre-existing permanent cardiac lead or lead system. During or after implantation of the implantable cardiac lead(s), an external pacing system analyzer, e.g., the MEDTRONIC® Model No. 5311B PSA, is attached to the proximal lead connector end assembly accessible through the incision to assess the performance of the system. Again, the Model 5311B connector elements are not compatible with the lead connector end elements for safety reasons. It is necessary to use either a disposable or a reusable “surgical cable” adaptor to complete the connection. Examples include the MEDTRONIC® Model 5410/S reusable surgical cable and the combination of the MEDTRONIC® Model 5105/S reusable adaptor and Model 5833/S disposable surgical cable.

Some of the above-identified patient and surgical cable adaptors constitute simply a connector assembly at one end that is compatible with the PSA or temporary pacemaker terminals, an external lead body enclosing the external lead conductors, and lead connector element connectors at the other end. Typically, two to four conductors are included in the lead body, and a set of two or four alligator clips are provided at the other ends for attachment to the lead connector ring and pin of one or two implantable cardiac leads.

Other, typically reusable patient and surgical cables employ a mechanism for receiving the proximal connector ends of the heart wire, temporary pacing lead or permanent cardiac lead. For example, either RCA type female sockets are used or a dual thumb screw attachment mechanism is used in the above-referenced MEDTRONIC® Model 5832/S and Model 5433A/V reusable patient cables, respectively.

Commonly assigned U.S. Pat. Nos. 4,245,642 and 4,466,441 disclose medical lead adaptors of the latter type wherein lead connector end assemblies are insertable into sockets of a housing to make electrical contact with a single or two electrical contacts spaced apart therein to receive bifurcated bipolar, in-line bipolar, or unipolar lead connector rings and/or pins. The attachment is effected by tightening down thumbscrews to prevent the connector end assemblies from slipping out of the socket openings of the housing in each case. It is not possible to make an attachment with a permanent pacing lead having a stylet wire fitted within the lead lumen and projecting out proximally through the connector pin. This case can only be accommodated by the use of alligator clips that attach across the connector rings and pins.

Such an attachment is not as secure and electrically isolated as would be desirable. It is undesirable to either lose the connection or to allow an electrical static discharge or other shock or impulse to reach the heart through the exposed lead connector ends. At present, it is necessary to loop and tape the assembled adaptor lead and implantable lead body against the patient's body and also place tape over the alligator clips.

At times, it has been observed that the careless use of alligator clips can damage the insulation sheathes adjacent to the lead connector end ring or connector pins. In IS-1 leads, damage to the insulating sheath and the seal rings on either side of the connector ring has been observed due to movement of the jaws of the alligator clips. Commonly assigned U.S. Pat. Nos. 6,192,278, 6,038,481, 6,038,479 and 5,931,861, incorporated herein by reference, describe a medical lead adaptor that provides a rapid, secure, insulated connection of the lead connector end assembly of a cardiac lead having electrodes adapted to be placed in or on the body with an external device, with a locking mechanism that holds a lead connector ring and/or pin in contact with ring and/or pin receptacle contacts.

### SUMMARY OF THE INVENTION

It is an object of the present invention to solve these problems identified with prior art methods and mechanisms for attaching an external medical device to a cardiac lead of the types including temporary pacing leads and heart wires of the types having unipolar lead connector end assemblies or bipolar, in-line, lead connector end assemblies.

According to a preferred embodiment of the present invention, in a medical lead adaptor assembly for making an electrical connection between a cardiac lead and an external medical device, the cardiac lead including a lead connector pin for electrically coupling the cardiac lead to the lead adaptor assembly, the lead adaptor assembly includes a connector end assembly electrically coupling the lead adaptor assembly to the external device. A housing portion of the lead adaptor assembly includes a lead receptacle portion including a lead receptacle that receives the lead connector pin of the cardiac lead to electrically couple the cardiac lead to the lead adaptor assembly, and a retaining flange extends from the housing portion and has a retaining slot that receives and retains the cardiac lead to substantially prevent corruption of the electrical coupling of the cardiac lead to the lead adaptor assembly.

According to a preferred embodiment of the present invention, a medical lead adaptor assembly system includes a lead adaptor assembly, a cardiac lead inserted within a patient and having a connector pin electrically coupling the cardiac lead and the lead adaptor assembly, an external medical device that performs cardiac stimulation and monitoring of the patient through the lead adaptor assembly and the cardiac lead, and a retaining flange, positioned on the lead adaptor assembly, having a retaining slot that receives and retains the cardiac lead to substantially prevent corruption of the electrical coupling of the cardiac lead and the lead adaptor assembly.

According to another preferred embodiment of the present invention, a medical lead adaptor assembly for making an electrical connection between a plurality of cardiac leads inserted within a patient and an external medical device includes a housing having a top portion and a bottom portion, a lead receptacle portion extending along a vertical plane extending between the top portion and the bottom portion, and an external device lead extending from the housing to a distal external connector end assembly electrically coupling the lead adaptor assembly to the external device. A first lead receptacle, positioned on the lead receptacle portion, receives a first connector pin of a first cardiac lead of the plurality of cardiac leads to electrically couple the first connector pin to the first lead receptacle to electrically couple the first cardiac lead to the external device through the lead adaptor assembly. A second lead receptacle, positioned on the lead receptacle portion, receives a second connector pin of a second cardiac lead of the plurality of cardiac leads to electrically couple the second connector pin to the second lead receptacle to electrically couple the second cardiac lead to the external device through the lead adaptor assembly. A retaining flange extends from bottom portion of the housing and has a first retaining slot that receives and retains the first cardiac lead and a second retaining slot that receives and retains the second cardiac lead, wherein the retaining flange extends from the bottom portion at an angle from the vertical plane to substantially prevent corruption of the electrical coupling of the first cardiac lead to the first lead receptacle and the second cardiac lead to the second lead receptacle.

### BRIEF DESCRIPTION OF THE DRAWINGS

The features of the present invention which are believed to be novel are set forth with particularity in the appended claims. The invention, together with further objects and advantages thereof, may best be understood by making reference to the following description, taken in conjunction with the accompanying drawings, in the several figures of which like reference numerals identify like elements, and wherein:

FIG. 1 is a simplified schematic view of a medical lead adaptor system according to a preferred embodiment of the present invention.

FIG. 2 is a schematic diagram of a medical lead adaptor assembly of the medical lead adaptor system of FIG. 1.

FIG. 3 is an isometric top view of a medical lead adaptor assembly according to a preferred embodiment of the present invention.

FIG. 4 is an isometric bottom view of the lead adaptor assembly of FIG. 3.

FIG. 5 is a front planar view of the medical lead adaptor assembly of FIG. 2.

FIG. 6 is a side planar view of the medical lead adaptor assembly of FIG. 2.

FIG. 7 is a schematic diagram of a medical lead adaptor assembly according to the present invention.

FIG. 8 is a top planar view of the medical lead adaptor assembly of FIG. 2.

FIG. 9 is a bottom planar view of the medical lead adaptor assembly of FIG. 2.

### DETAILED DESCRIPTION OF THE PREFERRED EMBODIMENT

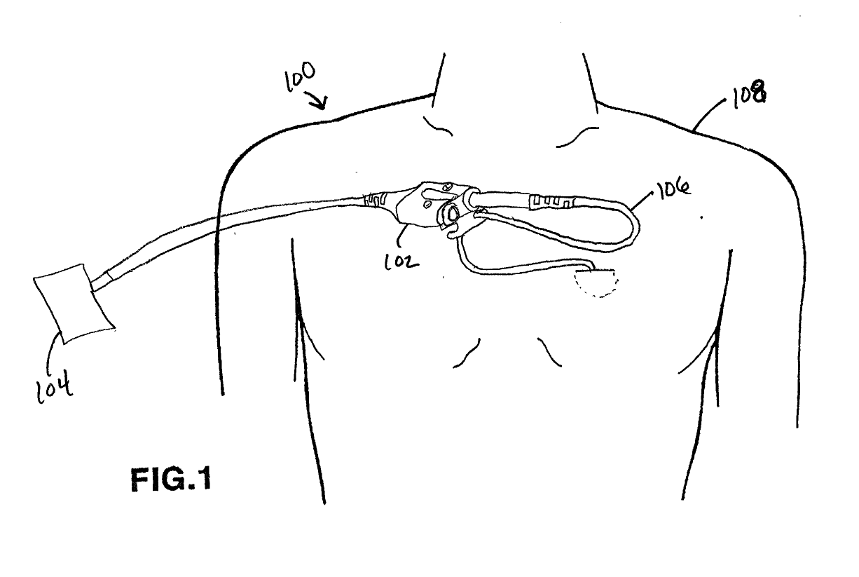

FIG. 1 is a simplified schematic view of a medical lead adaptor system according to a preferred embodiment of the present invention. As illustrated in FIG. 1, a medical lead adaptor system 100 according to a preferred embodiment of the present invention includes a medical lead adaptor assembly 102 for making a rapid electrical connection between one or more external medical device connection terminals (not shown) of an external medical device 104 for performing cardiac stimulation and monitoring, and one or more cardiac leads 106 inserted within a patient 108. The external medical device connection terminals may take any form, such as those associated with the above-referenced MEDTRONIC® Model 5311B PSA or Model 5348 and 5388 temporary pacemakers, for example.

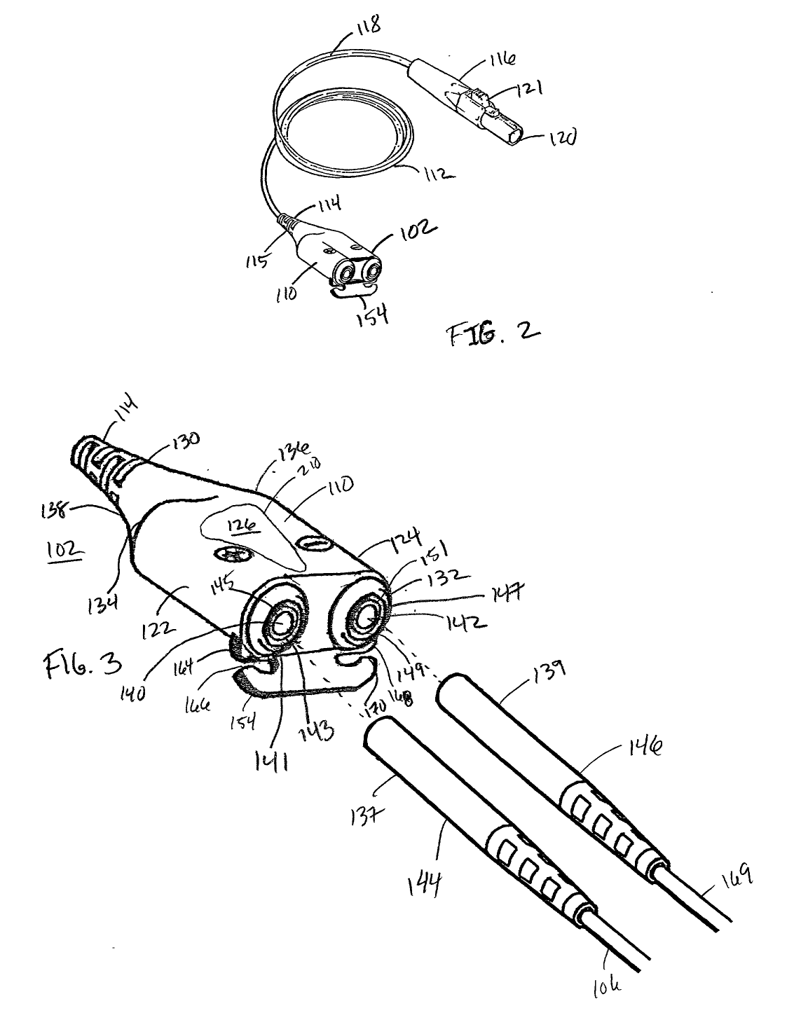

FIG. 2 is a schematic diagram of a medical lead adaptor assembly of the medical lead adaptor system of FIG. 1. As illustrated in FIGS. 1 and 2, lead adaptor assembly 102 includes a housing portion 110 and an external device lead 112. External device lead 112 extends from a proximal external lead conductor end portion 114 of housing 110 to a distal external lead connector end assembly 116 of any of the known types described above for making electrical contact with the one or more external medical device connection terminals of external medical device 104. External lead conductor end portion 114 is generally tubular in shape and includes spaced apart rails 115 extending around conductor end portion 114 to enable multidirectional movement of external device lead 112 relative to lead adaptor assembly 102.

According to a preferred embodiment of the present invention, external device lead 112 includes an external device lead body 118 that encases two electrically isolated external electrical conductors (not shown) of any known configuration that are coupled at respective ends with two external lead connector pins (not shown) located within external lead connector end assembly 116. The two external lead connector pins extend outward towards an opening 118 of external lead connector end assembly 116 in order to come in contact with the external medical device connection terminals of external medical device 104 when external lead connector end assembly 116 is inserted within external medical device 104, enabling lead adaptor assembly 102 and external medical device 104 to be electrically coupled along external device lead 112.

In addition, external lead connector end assembly 116 includes a locking mechanism 120 for fixedly engaging end assembly 116 within external medical device 104 to substantially substantially prevent inadvertent release of external lead connector end assembly 116 from external medical device 104, corrupting the electrical coupling between lead adaptor assembly 102 and external medical device 104, such as during patient movement or manipulation of lead adaptor assembly 102 and/or external medical device 104, for example.

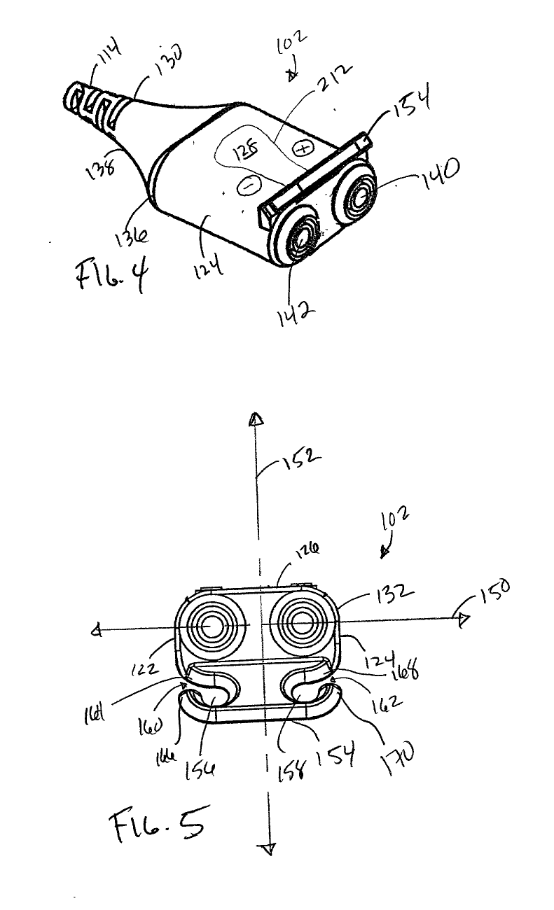

FIG. 3 is an isometric top view of a medical lead adaptor assembly according to a preferred embodiment of the present invention. FIG. 4 is an isometric bottom view of the lead adaptor assembly of FIG. 3. As illustrated in FIGS. 3 and 4, housing portion 110 of lead adaptor assembly 102 is generally longitudinal, having generally curved outer surface side portions 122 and 124 along with top and bottom portions 126 and 128 extending between a proximal end portion 130 of conductor end portion 114 and a lead receptacle portion 132. In particular, side portions 122 and 124 of lead adaptor assembly 102 extend from lead receptacle portion 132 to respective lead receptacle end portions 134 and 136, and from respective lead receptacle end portions 134 and 136 to proximal end portion 130 to form generally curved outer surfaces extending from lead receptacle portion 132 to proximal end portion 130. Top portion 126 and bottom portion 128 are generally equally spaced apart from each other along a portion of lead adaptor assembly 102 between lead receptacle portion 132 and proximal end portion 130. On the other hand, while side portion 122 and side portion 124 are generally equally spaced apart from each other along a portion of lead adaptor assembly 102 between lead receptacle portion 132 and respective lead receptacle end portions 134 and 136, the distance that side portion 122 and side portion 124 are spaced apart from each other decreases along a portion of lead adaptor assembly 102 between lead receptacle end portions 134 and 136 and proximal end portion 130 to form a generally angled portion 138 of lead adaptor assembly 102 along surface side portions 122 and 124 between lead receptacle end portions 134 and 136 and proximal end portion 130.

Two unipolar “negative” and “positive” lead receptacles 140 and 142 are formed within housing 110 of lead adaptor assembly 102 at lead receptacle portion 132. Lead receptacle 140 includes an outer portion 141 forming an opening 143. A connector ring 145 is centrally located within opening 143 to enable receptacle 140 to receive a shrouded portion of a unipolar connector pin of temporary pacing leads or heart wires, or at least one indifferent electrode bearing lead between connector ring 145 and outer portion 141. Similarly, lead receptacle 142 includes an outer portion 147 forming an opening 149. A connector ring 151 is centrally located within opening 149 to enable receptacle 142 to receive a shrouded portion of a unipolar connector pin of temporary pacing leads or heart wires, or at least one indifferent electrode bearing lead between connector ring 151 and outer portion 147. For example, a typical unipolar connector pin 144 (shown in FIGS. 1 and 3) of temporary pacing leads or heart wires of the type described above is inserted axially into unipolar receptacle 140, and unipolar connector pin 146 is inserted axially into unipolar receptacle 142 (only unipolar connector pin 144 is shown in FIG. 1). A shrouded portion 137 of connector pin 144 is received between connector ring 145 and outer portion 141 in opening 143 of lead receptacle 140 to enable an electrical connection to be formed between cardiac lead 106 and external device 104 through lead adaptor assembly 102. In the same way, a shrouded portion 139 of connector pin 146 is received between connector ring 151 and outer portion 147 in opening 149 of lead receptacle 142 to enable an electrical connection to be formed between cardiac lead 169 and external device 104 through lead adaptor assembly 102.

Top portion 126 and bottom portion 128 of lead adaptor assembly 102 are labeled “+” and “−” adjacent to unipolar receptacles 140 and 142, respectively. In such a use, both of the electrodes of the temporary leads or heart wires attached to the unipolar lead connector pins 144, 146 can be attached to the patient's heart for temporary pacing. All of the above-referenced MEDTRONIC heart wire and temporary pacing lead models other than the TEMPTRON temporary lead employ bifurcated lead connector end assemblies terminating in separated connector pins 144 and 146. However, an indifferent electrode on the patient's skin and connected to a connector pin of the same type can be substituted for one of the implanted bipolar heart wire or temporary pacing lead connector pins 144 or 146, depending on maintaining the proper polarity. Negative and positive unipolar receptacles 140 and 142, and corresponding unipolar openings 143 and 149 and connector rings 145 and 151, are sized in conjunction with contacts contained therein to accept the range of diameters of lead connector pins 144 and 146 in current use.

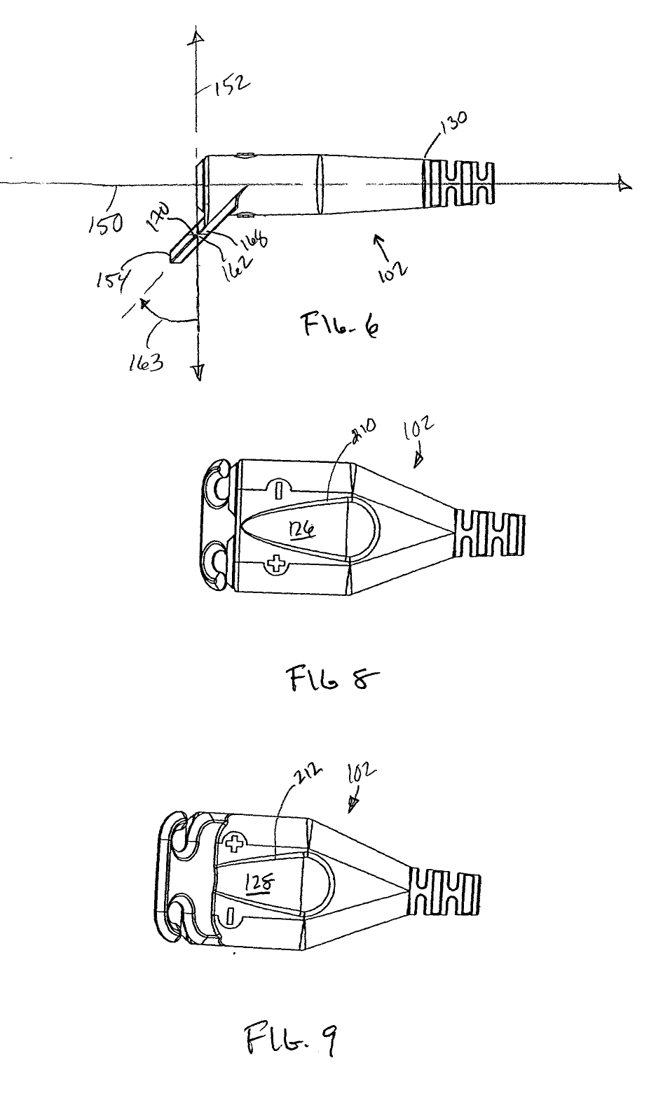

FIG. 5 is a front planar view of the medical lead adaptor assembly of FIG. 2. FIG. 6 is a side planar view of the medical lead adaptor assembly of FIG. 2. As illustrated in FIG. 5, lead receptacle portion 132 extends horizontally between side portion 122 and side portion 124 along a horizontal plane 150, and extends vertically between top portion 126 and bottom portion 128 along a vertical plane 152. As illustrated in FIGS. 2-6, a retaining flange 154 extends outward from lead receptacle portion 132 along bottom portion 128 of lead adaptor assembly 102, and includes one or more retaining slots 156 and 158, each having a slot opening 160 and 162, respectively. Retaining slots 156 and 158 are shaped internally in diameter to conformably receive and retain cardiac lead 106 when cardiac lead 106 is inserted within retaining slots 156 and 158 through slot opening 160 or slot opening 162.

As illustrated in FIGS. 3 and 6, slot opening 162 of retaining slot 158 is formed between an upper portion 164 and a lower tab portion 166. In the same way, slot opening 160 of retaining slot 156 is also formed between an upper portion 168 and a lower tab portion 170. According to the present invention, tab portion 164 is spaced from tab portion 166 at a distance less than the thickness of cardiac lead 106, and tab portion 168 is spaced from tab portion 170 at a distance less than the thickness of cardiac lead 169 (shown in FIG. 7). As illustrated in FIG. 6, retaining flange 154 is positioned at an angle 163 from vertical plane 152 to enable flange to substantially prevent lead connector pins 144 and 146 from being inadvertently removed from unipolar receptacles 140 and 142, respectively, located on lead receptacle portion 132 of lead adaptor assembly 102, as will be described below. According to a preferred embodiment of the present invention, angle 163 at which retaining flange 154 is positioned from vertical plane 152 is approximately equal to 45 degrees. However, it is understood that according to the present invention any desired value may be utilized for angle 163 that would enable retaining flange 154 to substantially prevent lead connector pins 144 and 146 from being inadvertently removed from unipolar receptacles 140 and 142, respectively. Furthermore, it is understood that retaining flange 154 may be adjustable so that angle 163 at which retaining flange 154 is positioned from vertical plane 152 may be variable.

FIG. 7 is a schematic diagram of a medical lead adaptor assembly according to the present invention. As illustrated in FIGS. 1 and 7, according to the present invention, once connector pin 144 of temporary pacing leads or heart wires of the type described above is inserted axially into unipolar receptacle 140, cardiac lead 106 of connector pin 144 is inserted within retaining slot 156 by being forcible inserted between tab portion 164 and tab portion 166 and into slot opening 160. Similarly, once connector pin 146 is inserted axially into unipolar receptacle 142, cardiac lead 169 of connector pin 146 is inserted within retaining slot 158 by being forcibly inserted between tab portion 168 and tab portion 170 and into slot opening 162. Since the distance between upper tab portion 164 and lower tab portion 166 and between upper tab portion 168 and lower tab portion 170 is less than the thickness of cardiac lead 106 and 169, respectively, cardiac lead 106 is forcible snapped between upper tab portion 164 and lower tab portion 166 in order to be inserted within retaining slot 156, and cardiac lead 169 is forcibly snapped between upper tab portion 168 and lower tab portion 170 in order to be inserted within retaining slot 158. In the same way, since the distance between upper tab portion 164 and lower tab portion 166 and between upper tab portion 168 and lower tab portion 170 is less than the thickness of cardiac lead 106 and 169, respectively, once cardiac leads 106 and/or 169 are inserted between upper tab portion 164 and lower tab portion 166 and into retaining slot 156, or between upper tab portion 168 and lower tab portion 170 and into retaining slot 158, cardiac leads 106 and 169 are inhibited from exiting retaining slots 156 and 158. However, once it is desirable to do so, cardiac leads 106 and 169 can be removed from retaining slots 156 and 158 by being forcible snapped between upper portion 164 and lower tab portion 166 and between upper portion 168 and lower tab portion 170, respectively, and outside of retaining slots 156 and 158.

As illustrated in FIG. 7, according to the present invention, by inserting cardiac leads 106 and 169 within retaining slots 156 and 158 of retaining flange 154 once connector pins 144 and 146 are inserted within receptacles 140 and 142, respectively, thereby electrically coupling cardiac leads 106 and 169 to external device 104, retaining flange 154 substantially prevents inadvertent removal of connector pins 144 and 146 from receptacles 140 and 142, or corruption of the electrical connection between connector pins 144 and 146 and receptacles 140 and 142 of lead adaptor assembly 102, such as during movement of the patient, external device 104, or lead adaptor assembly 102. For example, as illustrated in FIG. 7, according to the present invention, if an outward directed force, i.e., in a direction away from lead reception portion 132 of lead adaptor assembly 102, indicated by arrow 200, is exerted on cardiac lead 106 and/or on cardiac lead 169 as a result of movement of the patient, external device 104, or lead adaptor assembly 102, for example, retaining flange 154 reduces the effect of force 200 on connector pin 144 and/or connector pin 146. In particular, retaining flange 154 reduces the effect of force 200 exerted on one or both of connector pins 144 and 146 that would otherwise tend to have a tendency to pull connector pins 144 and 146 out of respective receptacles 140 and 142 by redirecting the outward directed force 200 on connector pins 144 and 146 so that outward force 200 results in an inward directed force, i.e., in a direction towards lead receptacle portion 132 of lead adaptor assembly 102, indicated by arrow 202, being exerted on connector pins 144 and 146.

In particular, as illustrated in FIG. 7, according to the present invention, once a force in a direction away from lead receptacle portion 132 of lead adaptor assembly 102, indicated by arrow 200, is exerted on cardiac lead 169, such as during movement of the patient, external device 104, or lead adaptor assembly 102, for example, cardiac lead 169 is directed through retaining slot 158 until a portion 206 of cardiac lead 169 between retaining flange 154 and conductor end portion 114 becomes taut so that the slack within portion 206 of cardiac lead 169 is removed. Once portion 206 of cardiac lead 169 becomes taut as a result of the resultant outward force exerted upon cardiac lead 169 away from lead receptacle portion 132, i.e., in a direction indicated by arrow 200, any subsequent resultant outward force in the direction indicated by arrow 200 that is exerted on cardiac lead 169, tending to pull cardiac lead 169 away from lead receptacle portion 132, results in inward force 202 being exerted on connector pin 146, forcing connector pin 146 towards lead receptacle portion 132. As a result, the outward force exerted on cardiac lead 169 causes connector pin 146 to be directed within receptacle 142 of lead receptacle portion 132, in direction of arrow 202, so that the outward force exerted on cardiac lead 169 in the direction of arrow 200 results in the opposite, inward directed force, i.e., in direction of arrow 202, to be exerted on connector pin 146, forcing connector pin 146 within receptacle 142. In this way, retaining flange 154 of the present invention transfers outward force 200 exerted on cardiac lead 169 to result in an inward force 202 being exerted on connector pin 146.

In the same way, according to the present invention, once a force in a direction away from lead receptacle portion 132 of lead adaptor assembly 102, indicated by arrow 200, is exerted on cardiac lead 106, such as during movement of the patient, external device 104, or lead adaptor assembly 102, for example, cardiac lead 106 is directed through retaining slot 156 until a portion 208 of cardiac lead 106 between retaining flange 154 and conductor end portion 114 becomes taut so that the slack within portion 208 of cardiac lead 106 is removed. Once portion 208 of cardiac lead 106 becomes taut as a result of the resultant outward force exerted upon cardiac lead 106 away from lead receptacle portion 132, i.e., in a direction indicated by arrow 200, any subsequent resultant outward force exerted on cardiac lead 106, tending to pull cardiac lead 106 away from lead receptacle portion 132, results in an inward force in the direction of arrow 202 to be exerted on connector pin 144, directing connector pin 144 towards lead receptacle portion 132. As a result, the outward force exerted on cardiac lead 106 causes connector pin 144 to be directed within receptacle 140 of lead receptacle portion 132, in direction of arrow 202 so that the outward force exerted on cardiac lead 106 in the direction of arrow 200 results in an opposite, inward directed force, i.e., in the direction indicated by arrow 202, to be exerted on connector pin 144, forcing connector pin 144 within receptacle 140. In this way, retaining flange 154 of the present invention transfers outward force 200 exerted on cardiac lead 106 to result in an inward force 202 being exerted on connector pin 144.

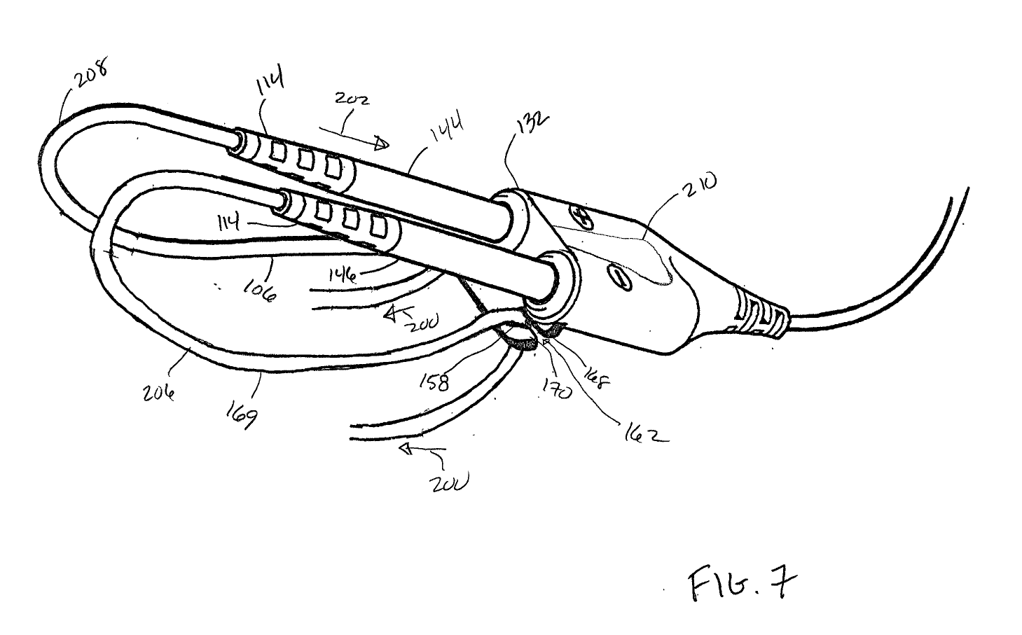

FIG. 8 is a top planar view of the medical lead adaptor assembly of FIG. 2 and FIG. 9 is a bottom planar view of the medical lead adaptor assembly of FIG. 2. As illustrated in FIGS. 3, 7 and 8, top portion 126 of housing portion 110 of lead connector assembly 102 includes a recessed portion 210 for placement of a finger of a user during handling of lead connector assembly 102, such as during insertion or removal of connector pins 144 and 146 from receptacles 140 and 142, respectively, or removal of cardiac leads 106 and 169 from retaining slots 156 and 158, respectively. Similarly, as illustrated in FIGS. 4 and 8, bottom portion 128 of lead connector assembly 102 includes a recessed portion 212 for placement of a finger of a user during handling of lead connector assembly 102, such as during insertion or removal of connector pins 144 and 146 from receptacles 140 and 142, respectively, or removal of cardiac leads 106 and 169 from retaining slots 156 and 158, respectively.

The preceding specific embodiments are illustrative of the practice of the invention. It is to be understood, therefore, that other expedients known to those of skill in the art or disclosed herein may be employed. In the following claims, means-plus-function clauses are intended to cover the structures described herein as performing the recited function and not only structural equivalents but also equivalent structures. For example, although a nail and a screw may not be structural equivalents in that a nail employs a cylindrical surface to secure wooden parts together, whereas a screw employs a helical surface, in the environment of fastening wooden parts, a nail and a screw are equivalent structures. It is therefore to be understood, that within the scope of the appended claims, the invention may be practiced otherwise than as specifically described without actually departing from the spirit and scope of the present invention.

What is claimed is:

1. A medical lead adaptor assembly for making an electrical connection between a cardiac lead and an external medical device, the cardiac lead including a lead connector pin for electrically coupling the cardiac lead to the lead adaptor assembly, the lead adaptor assembly comprising:
a connector end assembly electrically coupling the lead adaptor assembly to the external device;
a housing portion having a lead receptacle portion including a lead receptacle receiving the lead connector pin of the cardiac lead to electrically couple the cardiac lead to the lead adaptor assembly; and
a retaining flange extending from the housing portion and having a retaining slot receiving and retaining the cardiac lead to substantially prevent corruption of the electrical coupling of the cardiac lead to the lead adaptor assembly.

2. The medical lead adaptor assembly of claim 1, wherein the connector pin includes a shrouded portion, and wherein the lead receptacle includes an outer portion forming an opening and a connector ring centrally located within the opening to enable the lead receptacle to receive the shrouded portion of the connector pin between the connector ring and the outer portion.

3. The medical lead adaptor assembly of claim 1, wherein the retaining slot includes a slot opening, and wherein the retaining flange includes an upper portion spaced a distance from a lower tab portion to form the slot opening, the distance being less than a thickness of the cardiac lead.

4. The medical lead adaptor assembly of claim 1, wherein the housing portion includes a top portion and a bottom portion, the lead receptacle portion extending along a vertical plane between the top portion and the bottom portion, and wherein the retaining flange is positioned at an angle from the vertical plane to substantially prevent inadvertent removal of the lead connector pin from the lead receptacle.

5. The lead adaptor assembly of claim 4, wherein the angle is approximately equal to 45 degrees.

6. The lead adaptor assembly of claim 4, wherein the retaining flange extends outward along the bottom portion of the lead adaptor assembly.

7. The lead adaptor assembly of claim 1, wherein the retaining flange reduces the effect on the connector pin of a force exerted on the cardiac lead in a direction away from the lead reception portion.

8. The lead adaptor assembly of claim 1, wherein the retaining flange transfers an outward force exerted on the cardiac lead to result in an inward force being exerted on the connector pin.

9. The lead adaptor assembly of claim 1, wherein the housing portion includes a recessed portion for use during handling of the lead adaptor assembly.

10. A medical lead adaptor assembly system, comprising:
a lead adaptor assembly;
a cardiac lead inserted within a patient and having a connector pin electrically coupling the cardiac lead and the lead adaptor assembly;
an external medical device performing cardiac stimulation and monitoring of the patient through the lead adaptor assembly and the cardiac lead; and
a retaining flange, positioned on the lead adaptor assembly, having a retaining slot receiving and retaining the cardiac lead to substantially prevent corruption of the electrical coupling of the cardiac lead and the lead adaptor assembly.

11. The medical lead adaptor assembly system of claim 10, wherein the lead adaptor assembly further comprises:
a housing portion including a lead receptacle portion, the housing having a top portion and a bottom portion; and
a lead receptacle, positioned on the lead receptacle portion, receiving the connector pin to electrically couple the cardiac lead with the lead adaptor assembly; wherein the retaining slot includes a slot opening, and wherein the retaining flange includes an upper portion spaced a distance from a lower tab portion to form the slot opening, the distance being less than a thickness of the cardiac lead.

12. The medical lead adaptor assembly of claim 11, wherein the connector pin includes a shrouded portion, and wherein the lead receptacle includes an outer portion forming an opening and a connector ring centrally located within the opening to enable the lead receptacle to receive the shrouded portion of the connector pin between the connector ring and the outer portion.

13. The medical lead adaptor assembly system of claim 11, wherein the lead receptacle portion extends along a vertical plane between the top portion and the bottom portion of the housing, and wherein the retaining flange is positioned at an angle from the vertical plane to substantially prevent inadvertent removal of the lead connector pin from the lead receptacle.

14. The lead adaptor assembly system of claim 13, wherein the angle is approximately equal to 45 degrees.

15. The lead adaptor assembly system of claim 13, wherein the retaining flange extends outward along the bottom portion of the lead adaptor assembly.

16. The lead adaptor assembly system of claim 11, wherein the retaining flange reduces the effect on the connector pin of a force exerted on the cardiac lead in a direction away from the lead reception portion.

17. The lead adaptor assembly system of claim 10, wherein the retaining flange transfers an outward force exerted on the cardiac lead to result in an inward force being exerted on the connector pin.

18. The lead adaptor assembly system of claim 11, wherein the housing portion includes a recessed portion for use during handling of the lead adaptor assembly.

19. A medical lead adaptor assembly for making an electrical connection between a plurality of cardiac leads inserted within a patient and an external medical device, each of the plurality of cardiac leads including a lead connector pin for electrically coupling the plurality of cardiac leads to the lead adaptor assembly, the lead adaptor assembly comprising:
a housing having a top portion and a bottom portion;
a lead receptacle portion extending along a vertical plane extending between the top portion and the bottom portion;
an external device lead extending from the housing to a distal external connector end assembly electrically coupling the lead adaptor assembly to the external device;
a first lead receptacle, positioned on the lead receptacle portion, receiving a first connector pin of a first cardiac lead of the plurality of cardiac leads to electrically couple the first connector pin to the first lead receptacle to electrically couple the first cardiac lead to the external device through the lead adaptor assembly;
a second lead receptacle, positioned on the lead receptacle portion, receiving a second connector pin of a second cardiac lead of the plurality of cardiac leads to electrically couple the second connector pin to the second lead receptacle to electrically couple the second cardiac lead to the external device through the lead adaptor assembly;
a retaining flange extending from bottom portion of the housing and having a first retaining slot receiving and retaining the first cardiac lead and a second retaining slot receiving and retaining the second cardiac lead, wherein the retaining flange extends from the bottom portion at an angle from the vertical plane to substantially prevent corruption of the electrical coupling of the first cardiac lead to the first lead receptacle and the second cardiac lead to the second lead receptacle.

20. The lead adaptor assembly of claim 19, wherein the first retaining slot includes a first slot opening and the second retaining slot includes a second slot opening, and wherein the retaining flange includes a first upper portion spaced a first distance from a first lower tab portion to form the first slot and a second upper portion spaced a second distance from a second lower tab portion to form the second slot, the first distance being less than a thickness of the first cardiac lead and the second distance being less than a thickness of the second cardiac lead to fixedly retaining the first cardiac lead and the second cardiac lead within the first slot and the second slot, respectively.

21. The lead adaptor assembly of claim 20, wherein the angle is approximately equal to 45 degrees.

22. The lead adaptor assembly of claim 21, wherein the retaining flange reduces the effect on the first connector pin and the second connector pin of a force exerted on the first cardiac lead and the second cardiac lead, respectively, in a direction away from the lead reception portion.

23. The lead adaptor assembly of claim 21, wherein the retaining flange transfers an outward force exerted on the first cardiac lead and on the second cardiac lead to result in an inward force being exerted on the first connector pin and on the second connector pin.

24. The lead adaptor assembly of claim 21, wherein the housing includes a recessed portion for use during handling of the lead adaptor assembly.
